SEQUENCE LISTING (1 sequence)

SEQ ID NO: 1 - protein, 262 aa, Thermococcus kodakarensis
MGSSHHHHHHMSKDKVTVITSPSTEELVSLVNSALLEEAMLTIFARCKVHYDGRAKSELGSGDRVIIVKPDGSFLIHQSKKREPVNWQPPGSRVRLELRENPVLVSIRRKPRETLEVELEEVYMVSVFRAEDYEELALTGSEAEMAELIFENPEVIEPGFKPLFREKAIGTGIVDVLGRDSDGNIVVLELKRRRAELHAVRQLKSYVEILREEYGDKVRGILVAPSLTSGAKRLLEKEGLEFRKLEPPKRDSKKKGRQKTLF

(19) United States
(12) Patent Application Publication
(10) Pub. No.: US 2024/0076713 A1
(43) Pub. Date: Mar. 7, 2024

(54) Methods for Detecting Site-Specific and Spurious Genomic Deamination Induced by Base Editing Technologies

(71) Applicant: The General Hospital Corporation, Boston, MA (US)

(72) Inventors: J. Keith Joung, Winchester, MA (US); James Angstman, Charlestown, MA (US); Jason Michael Gehrke, Cambridge, MA (US)

(21) Appl. No.: 18/339,788

(22) Filed: Jun. 22, 2023

Related U.S. Application Data
(62) Division of application No. 16/754,648, filed on Apr. 8, 2020, now Pat. No. 11,725,228.
(60) Provisional application No. 62/571,222, filed on Oct. 11, 2017.

(51) Int. Cl. C12Q 1/44; C12Q 1/6855; C12Q 1/6869

(52) U.S. Cl. CPC C12Q 1/44; C12Q 1/6855; C12Q 1/6869; C12Q 2521/301; C12Q 2523/301; C12Q 2525/191; C12Q 2531/113

(57) ABSTRACT

Methodologies to detect off-target mutations induced by the deaminase activity of Base Editing technology.

### CLAIM OF PRIORITY

This application is a divisional of U.S. patent application Ser. No. 16/754,648, filed Apr. 8, 2020, which is a § 371 National Stage Application of PCT/US2018/055406, filed Oct. 11, 2018, which claims the benefit of U.S. Provisional Application Ser. No. 62/571,222, filed Oct. 11, 2017. The entire contents of the foregoing are incorporated herein by reference.

### FEDERALLY SPONSORED RESEARCH OR DEVELOPMENT

This invention was made with Government support under Grant Nos. GM118158 and HG009490 awarded by the National Institutes of Health. The Government has certain rights in the invention.

### SEQUENCE LISTING

This application contains a Sequence Listing that has been submitted electronically as an XML file named “29539-0306002_SL_ST26.XML.” The XML file, created on Jun. 21, 2023, is 2,225 bytes in size. The material in the XML file is hereby incorporated by reference in its entirety.

### TECHNICAL FIELD

Described herein are methodologies to detect off-target mutations induced by the deaminase activity of Base Editing technology.

### BACKGROUND

Base editing (BE) technologies use an engineered DNA binding domain such as zinc finger arrays or RNA-guided, enzymatically inactivated or deficient DNA binding protein such as nickase Cas9 (nCas9) to recruit a cytidine deaminase domain to a specific genomic location to effect site-specific cytosine-thymine transition substitutions^{1,2}.

### SUMMARY

The present invention is based on the development of methodologies to detect off-target mutations induced by the deaminase activity of Base Editing technology. Two methods are described. The first involves an in vitro Base-Editor targeting and deamination reaction of representative DNA populations followed by an enzymatic digestion specific to G:U mismatches and sequencing of the digested species, which will yield data on the types of targets that the full Base Editor enzyme can target and deaminate. The second is a targeted PCR-based enrichment protocol that will allow for the selective amplification of rare, yet important, genomic deamination-induced mutations that could arise from Base Editor's deaminase domain acting independently of any site-specific targeting by its engineered DNA binding domain. We also claim that this last technology may be further applicable to sense rare deletion events mediated by traditional double-stranded-break-inducing genome-editing nuclease technologies.

Thus, provided herein are methods for detecting deaminated sites in substrate DNA. The methods include providing a sample comprising substrate DNA, e.g., genomic DNA (gDNA) or synthesized DNA; deaminating the substrate DNA using a base editing fusion protein comprising a deaminase domain and a DNA binding domain, e.g., a zinc-finger domain; a transcription-activator-like effector domain; or a catalytically-inactive Cas9 or Cpf1, with a selected guide RNA, e.g., an sgRNA of interest; contacting the deaminated substrate with Endonuclease MS from Thermococcus kodakarensis (TkoEndoMS) to induce double strand breaks (DSBs) at deamination sites in the substrate DNA to produce DNA fragments with single-stranded, 5 base pair overhanging ends centered at the deamination site; treating the DNA fragments with uracil DNA glycosylase and endonuclease VIII to remove the deoxyuracil base from the ends of the DNA fragments; end-repairing and/or A-tailing the ends of the DNA fragments; and ligating an adapter oligonucleotide (preferably comprising sequences for use in high throughput sequencing) to the end; and sequencing the DNA fragments.

Also provided herein are methods for detecting deaminated sites in substrate DNA. The methods include providing a sample comprising substrate DNA; deaminating the substrate DNA using a base editing fusion protein comprising a deaminase domain, e.g., genomic DNA (gDNA) or synthesized DNA, and a nicking Cas9 protein (nCas9); contacting the deaminated substrate DNA with uracil DNA glycosylase and endonuclease VIII, to induce DSBs; end-repairing and/or A-tailing the ends of the DNA fragments; ligating adapter oligonucleotide (comprising sequences for use in high throughput sequencing) to the end; and sequencing the DNA fragments.

In some embodiments of the methods described herein, the adapter oligonucleotide comprises a single deoxyuridine, e.g., as described in US PG Pub. 2017/0088833. In some embodiments of the methods described herein, the adapter oligonucleotides comprise PCR primer binding sequences, and the methods comprises using PCR to enrich for sites that produced a DSB.

In some embodiments of the methods described herein, sequencing the DNA fragments comprises determining a sequence of at least about (i.e., ±10%) 10, 15, 20, 30, 50, 100, 150, 200, 250, 500, or more nucleotides at the ends of the DNA fragments.

In addition, provided herein are methods for detecting and quantifying base editor-induced cytosine to thymine mutation events in living cells. The methods include providing a sample comprising substrate genomic DNA from cells exposed to a base editor protein comprising a deaminase domain fused to DNA binding domain, e.g., a zinc-finger domain; a transcription-activator-like effector domain; or a Cas9 or Cpf1 nickase or catalytically-inactive Cas9 or Cpf1, with a selected guide RNA, e.g., an sgRNA of interest; using 3D PCR to selectively amplify alleles that have undergone deamination events, to create a population of amplicons that is enriched for deaminated alleles; and sequencing the enriched population of amplicons, preferably using next generation sequencing or TOPO cloning, to determine the identity of the amplified molecules. In some embodiments of the methods described herein, using 3D PCR to selectively amplify alleles that have undergone deamination events comprises shearing the substrate genomic DNA; ligating barcoded common adapters to the free ends of the sheared genomic DNA; and amplifying sites of interest with 3D PCR using one site-specific primer and one adapter-specific primer.

In some embodiments, the substrate genomic DNA is sheared randomly or semi-randomly using sonication or enzymatic treatment.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Unbiased, genome-wide in vitro method for discovering all off-target deamination events induced by BEs. Briefly, sheared genomic DNA (gDNA) is ligated to hairpin adaptors at both ends. After treatment with Lambda exo/ExoI to remove gDNA fragments, the hairpin adaptors are opened via treatment with USER enzyme to excise an internal uracil base and the resulting single stranded ends are used to circularize the gDNA-adaptor fragments via ligation. Treatment with Plasmid-Safe DNase removes all uncircularized DNA, leaving a homogenous population of circular gDNA fragments with a mean length of 300 base pairs. This population is then treated with purified BE protein in complex with an sgRNA of interest for two 20 hours at 37C to induce deamination at on- and off-target sites. Treatment of the deaminated DNA with TkoEndoMS creates DNA double strand breaks at all deamination sites. A second treatment with USER enzyme removes all BE-induced uracils, and DNA end repair and A-tailing produce suitable substrates for ligation to high throughput sequencing adaptors at each deamination site. PCR product enriched for on- and off-target site deamination events is subjected to high throughput sequencing and bioinformatic characterization to discover all in vitro deamination sites. Note that this strategy can also be adapted to use a nicking base editor protein. In this case, a DSB is generated by treatment with USER enzyme following deamination of the substrate DNA.

FIG. 2. Capillary electrophoresis data from an experiment demonstrating the specificity of TkoEndoMS' endonuclease activity for G:U DNA mismatches in vitro.

FIG. 3. 3D PCR uses a gradient of denaturation temperatures in otherwise standard PCR cycles applied across a 96-well plate. Each row contains 12 replicates of the same sample, and the left-most sample that successfully amplifies contains a library of PCR products derived from deaminated genomic DNA. The DNA in row A is unable to amplify at the same low temperatures because it does not contain deaminated genomic DNA.

FIG. 4. Schematic of modified 3D PCR with unique molecular indices to enable de-duplification of the high-throughput sequencing library. De-duplification allows for the bioinformatic determination of the total number of alleles with deamination events from the harvested genomic DNA.

### DETAILED DESCRIPTION

Base editing (BE) technologies use an engineered DNA binding domain (such as RNA-guided, catalytically inactive Cas9 (dead Cas9 or dCas9), a nickase version of Cas9 (nCas9), or zinc finger (ZF) arrays) to recruit a cytidine deaminase domain to a specific genomic location to effect site-specific cytosine-thymine transition substitutions^{1,2}; see also U.S. Ser. No. 62/541,544, which is incorporated herein by reference.

Since the deaminase domains used in BEs preferentially act on single-stranded DNA (ssDNA) substrates, it is thought that BEs that use nCas9 to direct their genomic targeting will be the most efficient configuration because nCas9-targeting involves the hybridization of a short guide RNA (sgRNA) to its genomic target and the concurrent displacement of the non-target strand into ssDNA in an R-loop. Like any other genome-editing technology, fully understanding a BE's ability to induce off-target mutations is an important step toward developing it for clinical applications; however, the subtle nature of the C→T mutation and the sheer number of cytosines that exist in the genome make BE's off-target mutations more difficult to detect than the off-target mutations induced by other kinds of genome-editing technologies.

With first-generation BE technology, we have identified three major potential sources of off-target mutagenesis. First, BE nCas9-stimulated R-loop formation can expose a total of 8 on-target nucleotides for deamination (5 of which having more-or-less equivalent propensities for mutagenesis) even though it may sometimes be necessary to restrict BE's mutagenic potential to only one target cytosine at a time. Second, Cas9 has a well-documented ability to bind at off-target sites with varying degrees of homology to its sgRNA³⁻⁴, which could lead to off-target R-loop formation and subjection of non-target cytosines to deamination. Third, BE's deaminase component could act upon naturally occurring genomic ssDNA or RNA substrates independent of Cas9-mediated targeting, resulting in spurious deamination.

Recently, a group described an in vitro strategy to evaluate off-target activities of BEs at sites which were both deaminated by the DA domain and nicked by nCas9s. Because it fails to detect sites which have been deaminated but not nicked, this strategy incompletely describes off-target deamination sites for these technologies, and has no ability to detect mutations that derive from spurious deamination. In addition, this method's general insensitivity and requirement for whole genome sequencing at 30-40× coverage for each sgRNA assessed makes it too cumbersome and expensive to be reasonably performed by most research laboratories and/or companies who may need to assess many BE:sgRNA complexes simultaneously. Here, we describe two new highly sensitive BE off-target deamination detection methodologies that will collectively enable the assessment of this critical parameter necessary for maturing BE toward therapeutic relevance, where modification of millions to billions of cells might be necessary.

Method 1: Detecting Deamination Events Induced by BE Technologies at Off-Target nCas9 or dCas9 Sites

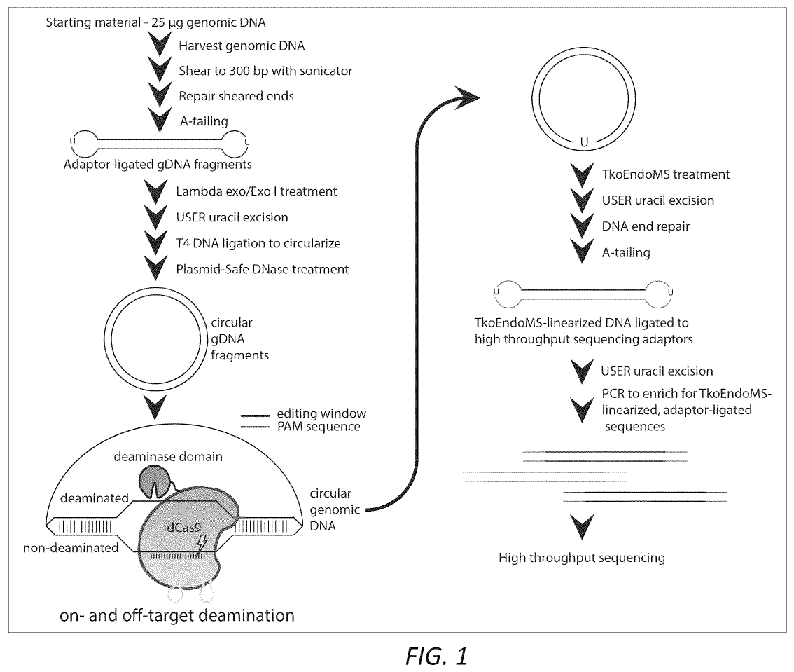

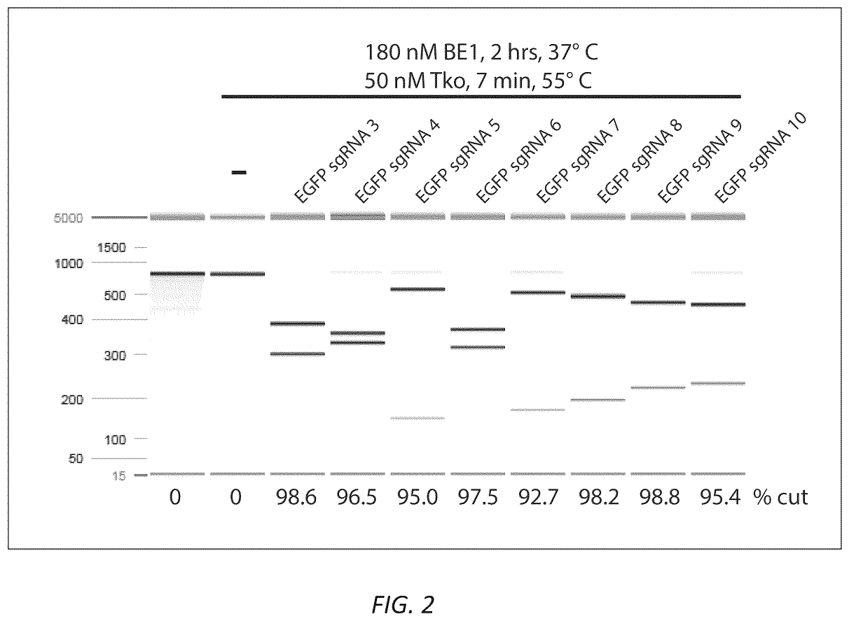

To describe all possible off-target deamination events induced by BE technologies in a sensitive and practical manner, an in vitro method modified from a previous in vitro assay for detecting off-targets of RNA-guided nucleases (CIRCLE-Seq⁶) can be used. This assay uses sheared, circularized genomic DNA (gDNA) or synthesized linear DNA as a reporter substrate for deamination events (FIG. 1). See also US PG Pub. 2017/0088833, which is incorporated herein in its entirety. In the modified assay, the substrate gDNA or synthesized DNA is deaminated by purified BE protein (consisting of a fusion protein between a deaminase domain and a DNA binding domain, e.g., a zinc finger, TALE, or catalytically-inactive Cas9 or Cpf1, e.g., as described in WO 2017/059313) in complex with an sgRNA of interest. TkoEndoMS, a protein derived from Thermococcus kodakarensis that has previously been shown to recognize G:T mismatches⁷ and that we have shown efficiently recognizes the G:U mismatch resulting from a BE deamination event (FIG. 2), is then used to induce double strand breaks (DSBs) at all deamination sites in the substrate DNA. The DSB induced by TkoEndoMS in the substrate gDNA produces two ends that, after repair and A-tailing, are suitable substrates for ligation to high throughput sequencing adaptors, allowing for the capture, enrichment and sequencing of these sites. Because this strategy relies solely on the presence of a G:U mismatch, it will capture all deamination sites induced by BEs in the substrate DNA, resulting in a more complete activity profile of these technologies for use in research and therapeutics.

Alternatively, the substrate gDNA or synthesized DNA is deaminated by purified BE protein composed of a deaminase domain and a nicking Cas9 protein. A DSB is then generated by treating the deaminated substrate DNA with USER enzyme (a mixture of uracil DNA glycosylase and endonuclease VIII), a reagent that specifically catalyzes the removal of uracil bases from DNA. This strategy produces two DNA ends that, following end repair, A-tailing, and ligation to high-throughput sequencing adapters, are compatible for PCR to enrich for sites that produced a DSB followed by deep sequencing.

Method 2: Detection of Spurious Deamination Events Induced by BE Technologies at Genomic Off-Target Sites Independent of nCas9 Targeting Using 3D PCR.

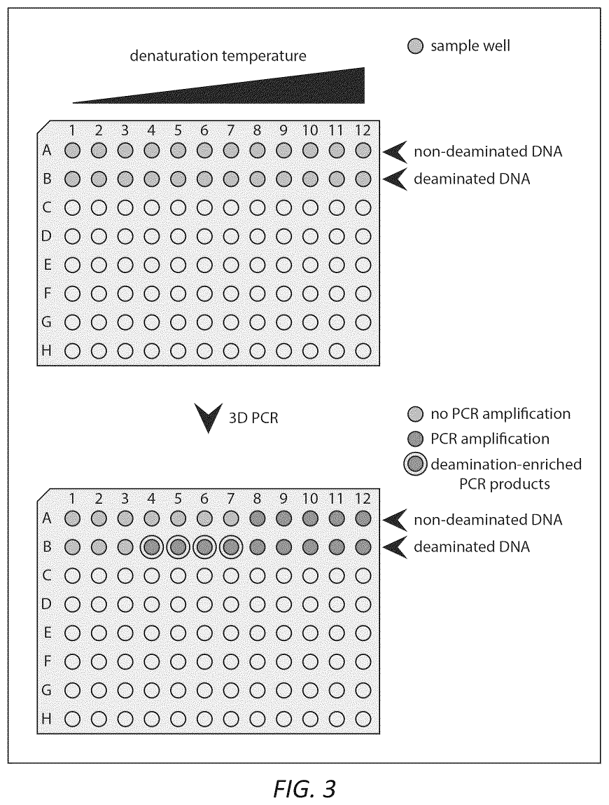

Because of the sheer number of cytosines in a genome, the relative odds that any given cytosine will be mutated by spurious deamination by BE technologies may remain extremely limited even if spurious deamination is a ubiquitous phenomenon with a high cumulative total of deamination events among all cells exposed to BE. Due to the error rate of Illumina sequencing methods, BE-induced mutation events that occur at a given genomic cytosine in fewer than 1 in ˜1000 cells will be undetectable by standard whole genome sequencing or PCR amplicon-based deep sequencing strategies³. One technique that has been described previously to enrich for extremely rare deamination events takes advantage of the differential DNA denaturation temperatures between deaminated amplicons containing slightly higher A:T content than their non-deaminated counterparts⁸⁻¹⁰. In this method, referred to as differential DNA denaturation PCR (3D PCR), the denaturation component of a standard PCR cycle is varied in a gradient across a row of PCR tubes or 96-well plate in order to find the lower limit for denaturation of the target amplicon. In complex samples containing very low numbers of amplicons in which deamination events have increased the A:T content, this allows for selective denaturation and exponential amplification of very rare deaminated amplicons (FIG. 3). The products from the lowest denaturation temperature 3D PCRs can then be subjected to high-throughput sequencing to determine which nucleotides within the amplicon have undergone C→T transition mutations. The sequencing can be done via a high-throughput strategy like Illumina sequencing, or using TOPO cloning, e.g., by inserting individual molecules from the 3D PCR population into plasmids and propagating those plasmids in bacteria, then sequencing those plasmids/inserts to determine the identity of the 3D PCR population of molecules.

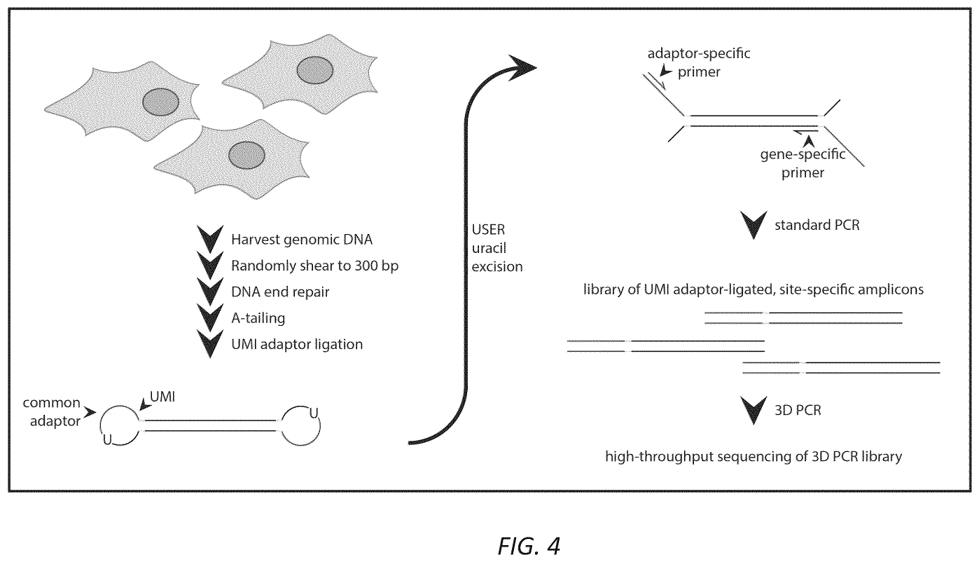

The 3D PCR method can selectively enrich for genomic sequences bearing C→T mutations and can be used to amplify and reliably detect rare deamination events in genomic DNA. However, PCR bias using current 3D PCR makes it difficult or impossible to determine the absolute rate of spurious deamination-mediated mutation events at a given locus. This is a critical parameter to quantify before any BE technologies can be deemed safe enough to use in therapeutic settings. Thus, the 3D PCR technique can be modified to selectively enrich for DNA amplicons that have undergone deamination events to assess the frequency of spurious deamination on a site-by-site basis. By creating 3D PCR substrates from the genomic DNA of deaminase-treated cells that has been randomly sheared and then ligated to hairpin adaptors containing a unique molecular index, we can quantify the number of alleles in a population that have undergone rare spurious deamination events by de-duplicating the reads obtained from high throughput sequencing of the 3D PCR library (FIG. 4).

While sampling issues may complicate this effort, careful determination as to the number of genomes' worth of DNA that are input into each UMI adaptor ligation and subsequent 3D PCR should yield a reasonably accurate number from which to calculate the spurious deamination rate. Others have previously calculated the enrichment factor of 3D PCR to be ˜10⁴, see ref¹¹, which would theoretically reduce the detection limit of spurious deamination events from ˜10⁻³ (from Illumina sequencing's error rate) to ˜10⁻⁷. Since a reasonable upper limit for the number of genomes input into a PCR is 10⁵ (approximately 400 ng of human gDNA), this method allows oversampling of any given deamination event by 100-fold. Simply dividing the number of distinct UMIs associated with reads containing C→T mutations in the 3D PCR Illumina sequencing data divided by the total number of UMIs observed in sequencing data of a parallel non-enriched PCR reaction on a similarly representative sample of DNA should yield the rate of BE-induced deamination events that occurred within that amplicon. We note that having enough unique UMIs such that there are ˜10× as many UMIs as possible genomic ligation partners will ensure a very low number of duplicate UMI usage (˜0.4% odds of any UMI being duplicated, by Poisson distribution) and therefore enable consistently precise calculations of the deamination error rates. Since a reasonable upper limit of input genomes is 10⁵, a 10 base pair random UMI containing 4×10¹⁰ unique members will almost always satisfy this condition.

Because the lower limit of template DNA denaturation must be determined empirically for each 3D PCR amplicon, the technique allows only for detection of spurious deamination at sites specified by the gene-specific primer in the 3D PCR reaction and may be difficult to scale to whole genome sequencing. However, we believe that by targeting genomic ‘hotspots’ known to be highly susceptible to deamination and/or sites that are particularly sensitive to spurious deamination¹²⁻¹³ (i.e., sites at which deamination results in oncogene expression), we can calculate an upper bound on genome-wide spurious deamination events and of spurious deamination events at sites which are likely to produce a disease phenotype.

This technique could be adapted to increase the sensitivity of on- and off-target detection for traditional nuclease-based genome-editing technologies. The in vitro discovery method of Cas9-mediated off-target mutagenesis called CIRCLE-Seq⁶ is thought to be able to discover the nearly complete suite of genomic off-target sites for to a given Cas9:sgRNA complex. However, validating off-target mutagenesis at sites where the mutation frequency is below 1 in ˜10,000 genomes has proven extremely challenging due to the intrinsic error rate of high throughput sequencing technology and the inability to enrich for these low frequency events in large populations. Therefore, some of the sites that CIRCLE-Seq identifies as off-targets are speculative and cannot be verified by targeted deep-sequencing. The combined UMI-ligation/3D PCR approach as described earlier in this section significantly improves on this 1 in ˜1000 detection limit. Since Cas9 frequently induces short deletions at on- and off-target sites, amplicons containing Cas9-mediated mutations can be enriched compared to unmodified DNA. Previous groups have reported that 1 C→T mutation per 250 base pairs of DNA causes a large enough differential PCR template denaturation temperature over unmodified DNA to become enriched in 3D PCR, so it stands to reason a 1 base pair deletion in 250 base pairs can also be enriched.¹⁴ In this scenario, the gene-specific priming will occur at a site predicted to be a Cas9 off-target site instead of one where we suspect spurious deamination has occurred. If the same enrichment factor of 10⁴ remains true of 3D PCR when used to selectively amplify sequences containing small deletions, the new off-target mutagenesis detection limit should be on the order of 10⁻⁷.

### Examples

The invention is further described in the following examples, which do not limit the scope of the invention described in the claims.

### Example 1. TkoEndoMS Recognizes G:U Mismatches Resulting from a BE Deamination Event

TkoEndoMS, a protein derived from Thermococcus kodakarensis, has previously been shown to recognize G:T mismatches⁷. A capillary electrophoresis experiment was performed to demonstrate the specificity of TkoEndoMS' endonuclease activity for G:U DNA mismatches in vitro. An 800 base pair PCR amplicon was incubated with purified BE protein and a variable sgRNA for two hours to induce site-specific deamination. After purification, the deaminated PCR amplicon was incubated with purified TkoEndoMS protein for 7 minutes to induce double strand breaks at G:U mismatches. The DNA was then separated by size by capillary electrophoresis and imaged. As shown in FIG. 2, TkoEndoMS efficiently recognized G:U mismatches resulting from a BE deamination event.

TkoEndoMS Sequence with N-Terminal Hexahistidine Tag for Protein Purification

(SEQ ID NO: 1)
MGSSHHHHHHMSKDKVTVITSPSTEELVSLVNSALLEEAMLTIFARCKVH
YDGRAKSELGSGDRVIIVKPDGSFLIHQSKKREPVNWQPPGSRVRLELRE
NPVLVSIRRKPRETLEVELEEVYMVSVFRAEDYEELALTGSEAEMAELIF
ENPEVIEPGFKPLFREKAIGTGIVDVLGRDSDGNIVVLELKRRRAELHAV
RQLKSYVEILREEYGDKVRGILVAPSLTSGAKRLLEKEGLEFRKLEPPKR
DSKKKGRQKTLF

### REFERENCES

1. Komor, Alexis C., Yongjoo B. Kim. Michael S. Packer, John A. Zuris, and David R. Liu. “Programmable Editing of a Target Base in Genomic DNA without Double-stranded DNA Cleavage.” Nature 533.7603 (2016): 420-24.
2. Yang, Luhan, Adrian W. Briggs, Wei Leong Chew, Prashant Mali, Marc Guell, John Aach, Daniel Bryan Goodman, David Cox, Yinan Kan. Emal Lesha, Venkataramanan Soundararajan, Feng Zhang, and George Church. “Engineering and Optimising Deaminase Fusions for Genome Editing.” Nature Communications 7 (2016): 13330.
3. Tsai, Shengdar Q., Zongli Zheng, Nhu T. Nguyen, Matthew Liebers, Ved V. Topkar, Vishal Thapar, Nicolas Wyvekens, Cyd Khayter, A. John Iafrate, Long P. Le, Martin J. Aryee, and J. Keith Joung. “GUIDE-seq Enables Genome-wide Profiling of Off-target Cleavage by CRISPR-Cas Nucleases.” Nature Biotechnology 33.2 (2014): 187-97.
4. Wu, Xuebing, David A. Scott, Andrea J. Kriz, Anthony C. Chiu, Patrick D. Hsu, Daniel B. Dadon, Albert W. Cheng, Alexandro E. Trevino. Silvana Konermann, Sidi Chen, Rudolf Jaenisch, Feng Zhang, and Phillip A. Sharp. “Genome-wide Binding of the CRISPR Endonuclease Cas9 in Mammalian Cells.” Nature Biotechnology 32.7 (2014): 670-76.
5. Kim, Daesik, Kayeong Linm, Sang-Tae Kim, Sun-Heui Yoon. Kyoungmi Kim. Seuk-Min Ryu, and Jin-Soo Kim. “Genome-wide Target Specificities of CRISPR RNA-guided Programmable Deaminases.” Nature Biotechnology (2017).
6. Tsai. Shengdar Q., et al. “CIRCLE-seq: a highly sensitive in vitro screen for genome-wide CRISPR-Cas9 nuclease off-targets.” Nature Methods (2017).
7. Ishino, Sonoko, et al. “Identification of a mismatch-specific endonuclease in hyperthermophilic Archaea.” Nucleic acids research (2016): gkw153.
8. Suspene. Rodolphe, et al. “Recovery of APOBEC3-edited human immunodeficiency virus G→A hypermutants by differential DNA denaturation PCR.” Journal of general virology 86.1 (2005): 125-129.
9. Aynaud, Marie-Ming, et al. “Human Tribbles 3 protects nuclear DNA from cytidine deamination by APOBEC3A.” Journal of Biological Chemistry 287.46 (2012): 39182-39192.
10. Shinohara, Masanobu, et al. “APOBEC3B can impair genomic stability by inducing base substitutions in genomic DNA in human cells.” Scientific reports 2 (2012): 806.
11. Suspene, Rodolphe, et al. “Extensive editing of both hepatitis B virus DNA strands by APOBEC3 cytidine deaminases in vitro and in vivo.” Proceedings of the National Academy of Sciences of the United States of America 102.23 (2005): 8321-8326.
12. Holtz, Colleen M., Holly A. Sadler, and Louis M. Mansky. “APOBEC3G cytosine deamination hotspots are defined by both sequence context and single-stranded DNA secondary structure.” Nucleic acids research (2013): gkt246.
13. Rebhandl, Stefan, Michael Huemer, Richard Greil, and Roland Geisberger. “AID/APOBEC Deaminases and Cancer.” Oncoscience 2 (2015): 320.
14. Suspène. R., V. Caval, M. Henry. M. S. Bouzidi, S. Wain-Hobson. and J-P Vartanian. “Erroneous Identification of APOBEC3-edited Chromosomal DNA in Cancer Genomics.” British Journal of Cancer 110.10 (2014): 2615-622.
15. Fu, Y., Sander. J. D., Reyon, D., Cascio, V. M. & Joung, J. K. Improving CRISPR-Cas nuclease specificity using truncated guide RNAs. Nature Biotechnology 32, 279-284 (2014).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-3. (canceled)

4. A method of detecting deaminated sites in substrate DNA, the method comprising:
providing a sample comprising substrate DNA;
deaminating the substrate DNA using a base editing fusion protein comprising a deaminase domain and a nicking Cas9 protein (nCas9);
contacting the deaminated substrate DNA with uracil DNA glycosylase and endonuclease VIII, to induce DSBs;
end-repairing and/or A-tailing the ends of the DNA fragments;
ligating adapter oligonucleotide (comprising sequences for use in high throughput sequencing) to the end; and
sequencing the DNA fragments.

5. The method of claim 4, wherein the substrate DNA is genomic DNA (gDNA) or synthesized DNA.

6. The method of claim 4, wherein the adapter oligonucleotides comprise PCR primer binding sequences, and the methods comprises using PCR to enrich for sites that produced a DSB.

7. The method of claim 4, wherein sequencing the DNA fragments comprises determining a sequence of at least about 10 or more nucleotides at the ends of the DNA fragments.

8. A method for detecting and quantifying base editor-induced cytosine to thymine mutation events in living cells, the method comprising:
providing a sample comprising substrate genomic DNA from cells exposed to a base editor protein comprising a deaminase domain fused to DNA binding domain, preferably a zinc-finger domain; a transcription-activator-like effector domain; or a Cas9 or Cpf1 nickase or catalytically-inactive Cas9 or Cpf1, with a selected guide RNA;
using 3D PCR to selectively amplify alleles that have undergone deamination events, to create a population of amplicons that is enriched for deaminated alleles; and
sequencing the enriched population of amplicons, preferably using next generation sequencing or TOPO cloning, to determine the identity of the amplified molecules.

9. The method of claim 8, wherein using 3D PCR to selectively amplify alleles that have undergone deamination events comprises:
shearing the substrate genomic DNA; ligating barcoded common adapters to the free ends of the sheared genomic DNA; and
amplifying sites of interest with 3D PCR using one site-specific primer and one adapter-specific primer.

10. The method of claim 9, wherein the substrate genomic DNA is sheared using sonication or enzymatic treatment.
